(19) United States
(12) Patent Application Publication
(10) Pub. No.: US 2020/0323499 A1
(43) Pub. Date: Oct. 15, 2020

(54) CONE BEAM CT MULTI-DIRECTIONAL SCANNING APPARATUS

(71) Applicant: XI'AN LIREN MEDICAL TECHNOLOGY CO., LTD, Shaanxi (CN)

(72) Inventors: Limin ZHAO, Xi'an (CN); Binxia HAN, Xi'an (CN); Jianhui ZHAO, Xi'an (CN)

(21) Appl. No.: 16/626,484

(22) PCT Filed: Aug. 30, 2017
(86) PCT No.: PCT/CN2017/097308

§ 371 Date: Dec. 24, 2019

(30) Foreign Application Priority Data
Jun. 27, 2017 (CN) 201710502698.0
Jun. 27, 2017 (CN) 201720756746.4

(51) Int. Cl. A61B 6/00; A61B 6/02; A61B 6/03; A61B 6/04; G06T 7/00

(52) U.S. Cl. CPC A61B 6/4085; A61B 6/027; A61B 6/032; G06T 2207/10081; G06T 7/0012; G06T 2207/30008; A61B 6/0457

(57) ABSTRACT

Disclosed is a cone-beam CT multi-directional scanning apparatus which comprises a host machine frame, a cone-beam CT scanning device, a scanning frame and a revolving driving device, wherein the cone-beam CT scanning device is arranged on a body of the scanning frame; and the scanning frame is connected to the host machine frame through the revolving driving device, so that the scanning frame can turn over inside the host machine frame. Scanning on a human body at a vertical position can be implemented when the scanning frame is turned to be in a lying state; and scanning on a human body's lying position can be implemented when the scanning frame is turned to be in a standing state. The cone-beam CT multi-directional scanning apparatus provided by the invention changes the current situation that three-dimensional bone imaging data on a human body's upright position cannot be acquired, and also changes the current situation that image resolving precision on a CT imaging sagittal plane is relatively low. Three-dimensional cone-beam CT imaging and two-dimensional DR forming of a patient can be implemented on the standing position and the lying position, so that three-dimensional images of human bone system and soft tissues, with high spatial precision and high density precision can be acquired.

### TECHNICAL FIELD

The invention relates to the technical field of medical scanning equipment, in particular to a cone-beam CT multi-directional scanning apparatus.

### BACKGROUND

Spine surgery department and pain treatment department are the fastest growing departments in the past ten years. With changes in human lifestyle brought about by the development of technology, patients suffering from various pains are explosively increasing and become younger and younger in average age. Human neck, shoulder, waist, and leg pain, including pain in hands, arms, and feet, are caused in considerable part by the spine, intervertebral discs, bones, and joints of the body. In the modern clinical development, the research and application of skeletal structure and biomechanics are becoming the key and hot spots in clinical practice. Three-dimensional imaging and 3D printing technology have brought about welcome changes to clinical technology.

MRI (Magnetic Resonance Imaging) and CT, which are the main means of current imaging examinations, conduct examinations of patients normally in a lying or horizontal position. Such a way of examination cannot truly reflect the changes in spine biomechanics during standing and the changes in the internal and external structures of the spinal canal during the extension of the lumbar spine. Only DR can form a two-dimensional image in the standing position, which brings certain obstacles to clinical diagnosis. In addition, it is hard to obtain high-quality 3D images due to the low accuracy of the current CT axis image, which impacts and restrict the current clinical diagnosis and research development of the spine, joints and bones.

The advent of ESAOTE G-SCAN represents the development of magnetic resonance examination positions from lying to standing, and from full-featured to dedicated. Due to the dynamic changes of the soft tissue structure in the standing position, problems often hidden in the conventional supine position are found. Whilst the clinical application of G-SCAN has been widely recognized abroad and has achieved certain results, there are still some deficiencies in G-SCAN scanning. The image produced by G-SCAN has a worse quality and a longer scanning time due to the lower field strength of the open magnetic resonance scanner in the standing position compared with high-field MRI, and it is hard for the patient to maintain his/her posture in the standing position. Currentyly , studies of lumbar spine MRI in the standing position only focused on morphological manifestations. The concerned technology needs further improvement and development.

At present, the cone-beam CT (CBCT) is mainly used for image tracking and localization in oral and skull imaging and tumor radiotherapy. Oral and skull imaging and applications have been very mature, with the main features as follows: 1) Low radiation; 2) clear images, with the spatial resolution of 0.1mm available, which greatly exceeds the axial resolution of 0.3mm to 0.5mm of CT, with high-quality 3D skeletal images available; and 3) a low price which is much lower than CT and MRI. At present, the large-size dynamic imaging board technology has been very mature, which establishes a good foundation for whole body imaging.

### SUMMARY

In order to solve the technical problem of CT scan in the prior art that is difficult to truly reflect the spine biomechanical changes during standing, the technical solution of the present invention is as follows:

According to the present invention, a cone-beam CT multi-directional scanning apparatus comprises a host machine frame, a cone-beam CT scanning device, a scanning frame and a revolving driving device, wherein the cone-beam CT scanning device is arranged on a body of the scanning frame; and the scanning frame is connected to the host machine frame through the revolving driving device so that the scanning frame can turn over inside the host machine frame, wherein scanning on human body at a vertical position can be implemented when the scanning frame is turned to be under the lying state, and scanning on human body at a lying position can be implemented when the scanning frame is turned to be under the standing state.

In an embodiment, the scanning apparatus may comprise a lifting platform, wherein a lifting end of the lifting platform is connected to the scanning frame through the revolving driving device.

In an embodiment, the cone-beam CT scanning device comprises a rotary scanning table, a bulb tube, and a dynamic flat-panel detector, wherein a detection port is provided in the middle of the rotary scanning table, the rotary scanning table is arranged on the body of the scanning frame, and the bulb tube and the dynamic flat-panel detector are oppositely disposed on both sides of the detection port.

In an embodiment, the revolving driving device may comprise a revolving axis, a bearing, and a motor, wherein a power end of the revolving axis is connected to an output end of the motor, and the bearing is sleeved outside the revolving axis.

In an embodiment, the scanning apparatus may comprise a base and a base guide rail pair, wherein the base is connected to the host machine frame through the base guide rail pair so that the host machine frame slides along the base guide rail pair.

In an embodiment, the scanning apparatus may comprise a standing platform, wherein the standing platform is disposed below the detection port.

In an embodiment, the scanning apparatus may comprise a lifting scanning bed, wherein the width of the lifting scanning bed is smaller than the diameter of the detection port.

In an embodiment, the standing platform is a liftable platform.

Compared with the prior art, the cone-beam CT multi-directional scanning apparatus according to the present invention has the following beneficial effects.

The cone-beam CT multi-directional scanning apparatus provided by the invention makes it possible to obtain three-dimensional bone imaging data on a human body's upright position, and the image resolving precision of a CT imaging sagittal plane can be improved. Three-dimensional cone-beam CT imaging and two-dimensional DR forming can be implemented on human body at the standing position and the lying position, so that three-dimensional images of human bone system and soft tissues of high spatial precision and high density precision can be acquired, thereby providing good support for clinical biomechanical evaluation, diagnosis, treatment, surgical plan formulation, and simulation.

### BRIEF DESCRIPTION OF THE DRAWINGS

FIG. 1 is a side view of a cone-beam CT multi-directional scanning apparatus according to the present invention in use when a patient is in vertical position;

FIG. 2 is a top view of the cone-beam CT multi-directional scanning apparatus according to the present invention in use when a patient is in vertical position;

FIG. 3 is a top view of the cone-beam CT multi-directional scanning apparatus according to the present invention in use when a patient is in lying position;

FIG. 4 is a front view of the cone-beam CT multi-directional scanning apparatus according to the present invention in use when a patient is in lying position;

FIG. 5 is a side view of the cone-beam CT multi-directional scanning apparatus according to the present invention in use when a patient is in lying position.

### DETAILED DESCRIPTION

The technical solution of the present invention will be clearly and completely described below with reference to the accompanying drawings of the present invention. Based on the embodiments of the present invention, all other embodiments obtained by a person of ordinary skill in the art without inventive efforts shall fall within the protection scope of the present invention.

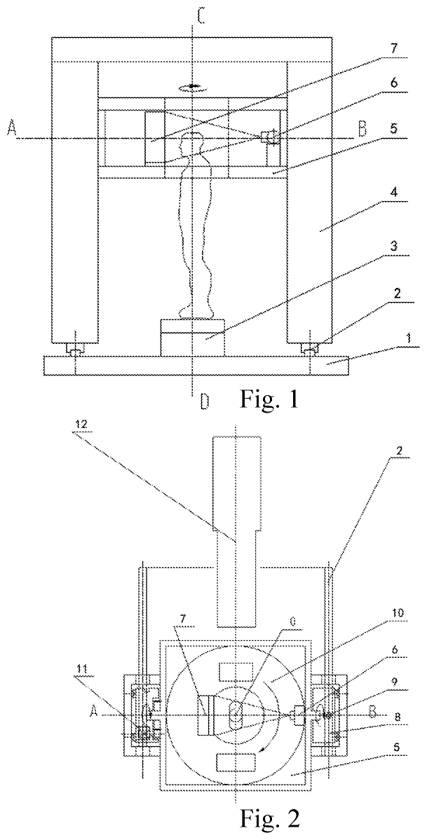

As shown in FIGS. 1 to 5, a cone-beam CT multi-directional scanning apparatus according to an embodiment of the present invention comprises a base 1 which is connect with a host machine frame 4 through a main base guide rail pair 2. The host machine frame 4 can move forward and backward along a guide rail in the main base guide rail pair 2 under the drive of a motor, so as to achieve a whole body scan and imaging of a patient in lying state.

A lifting platform is provided on two sides of the host machine frame 4, and a lifting end of the lifting platform is connected to the scanning frame by virtue of a revolving driving device. A cone-beam CT scanning device is arranged on the scanning frame 5. The lifting platform comprises a left lifting platform 8 and a right lifting platform 11. Both the left lifting platform 8 and the right lifting platform 11 are connected to the host machine frame 4 through a mechanical guiding and transmission driving device. A lifting end of the left lifting platform 8 is connected to the scanning frame 5 through a revolving axis, a bearing, and a motor. The right lifting platform 11 is connected to the scanning frame 5 through a revolving axis, a bearing, and a ball screw pair 9 driven by lifting. The scanning frame 5 and the rotary axis AB and the beam center axis of the left and right lifting platforms are at the same height. In this way, the scanning frame 5 can realize up and down movements on the host machine frame 4 to achieve the cone-beam CT scanning and imaging of the patient in standing state. It is also possible to achieve a turning by 90° around the axis AB front and back to perform the scan conversion between the standing state and the lying state of the patient.

The cone-beam CT scanning device comprises a rotary scanning table 10, a bulb tube 6, and a dynamic flat-panel detector 7. The rotary scanning table 10 is connected to the scanning frame 5 through a revolving bearing pair, a transmission drive device, and a conductive slip ring, etc. A detection port is opened in the center of the rotary scanning table 10. The bulb tube 6 and the dynamic flat-panel detector 7 are oppositely disposed on two sides of the detection port respectively. In order to avoid occurring of deviation in the positional relationship between the bulb tube 6 and the flat-panel detector 7 during the scanning process and to facilitate adjustment of relative position therebetween, a position adjustment mechanism is provided. The cone-beam CT scanning device is arranged on the rotary scanning table 10, so that the cone-beam CT scanning device can be rotated around the axis CD or original point o together with the rotary scanning table 10, thereby achieving rotation around the patient in the patient's standing position.

The function of the lifting platform described above is to enable the scanning frame 5 arranged thereon to move up and down during the scanning process of the patient in the standing state, so that a whole or partial body scanning of the patient in the standing position can be performed. The connection between the lifting platform and the scanning frame 5 is configured as a connection by virtue of the revolving driving device which can realize the rotation of the scanning frame 5 so that the working surface of the cone-beam CT scanning device on the scanning frame 5 can be set in the horizontal or vertical state, thereby facilitating the scanning and imaging of the patient while standing or lying.

In order to ensure the whole body scanning of the patient for a standing patient, a standing platform 3 is provided. The standing platform 3 is disposed below the detection port, so that when the cone-beam scanning of the patient in the standing state is performed, the patient is located at the center of the detection port, without affecting the detection effect. Meanwhile, the standing platform is configured as a liftable platform, so as to cooperate with the lifting platform connected to the scanning frame, so that in the process of cone-beam CT scanning, it is only necessary to set the movement trajectory and stroke of the lifting platform. As for the influence of each person's different height on the scanning, adjustment is performed only by virtue of the standing platform. This structure is simple and convenient and reduces the labor intensity of the medical staff.

In order to enable the scanning of the patient in the lying state, a lifting scanning bed 12 is provided. The lifting scanning bed 12 can be raised or lowered in the vertical direction. A horizontal slide rail is arranged below the lifting scanning bed 12, and the horizontal sliding rail can extend through the rotating scanning table in the vertical state, so that the lifting scanning bed 12 can slide in the horizontal direction. The width of the lifting scanning bed 12 is smaller than the diameter of the detection port. The scanning bed 12 is independently placed at the rear of the host machine frame 4 and can realize up, down, front and back movements, so as to meet the needs of the patient in the lying state to get on or out of bed and the need for scanning and imaging.

The cone-beam CT multi-directional scanning apparatus requires a control system, a data processing system, a host computer system, and an auxiliary system during use.

Control system is configured to control each of moving parts to operate according to the working logic flow of the apparatus, to control the operation of the flat-panel detector 7 synchronous with revolving of the rotary scanning table 10 and the strict cooperation between moving up and moving down of the scanning frame 5, to control the reading, transmission, and storage of data from the flat-panel detector 7, to control the data transmission of upper and lower computers, to conduct the detection and alarm of the equipment running status, to conduct the management and setting of equipment parameters, and to control manual operation switches, etc.

Data processing system is configured to process and calculate the acquired data of the flat-panel detector 7 according to a set program to generate graphic data files in a standard format.

Host computer system comprises customer information management, image data operation and output, backup, etc., and is configured to control some control devices of the host computer to work in accordance with the image scanning process.

Auxiliary system comprises a laser ruler for initial patient positioning, an intercom system inside and outside the computer room, a video surveillance system, etc.

### Embodiment 1

3D scanning and imaging of a patient in standing position:

As shown in FIGS. 1 and 2, the operator selects the upright 3D scanning after entering the basic patient information. With the confirmation of the auxiliary system, the patient is correctly standing on the standing platform 3, and the auxiliary system is used to define the required scanning examination range. After starting the examination, the apparatus operates according to the logical actions required by the control system. The rotary scanning table 10 rotates, and the bulb tube 6 emits a beam as required. The dynamic flat-panel detector 7 receives the projection information. The control system transmits data for saving. After a one cycle scanning, the scanning frame 5 is moved down by one imaging height for the next scanning, until the defined segments are scanned, and the machine returns to the initial state. Then the patient leaves the machine. After the data processing system processes the data to generate a data file, the operator outputs the corresponding data according to the examination requirements, which can be electronic data or film.

### Embodiment 2

3D scanning and imaging of a patient in lying position:

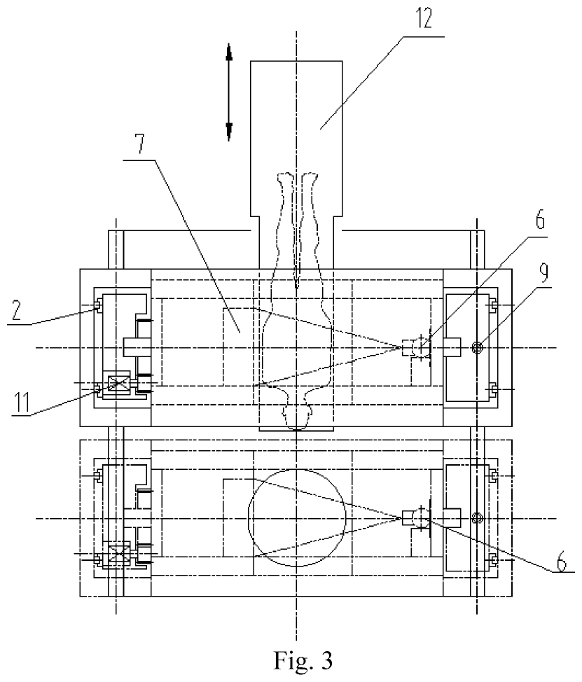

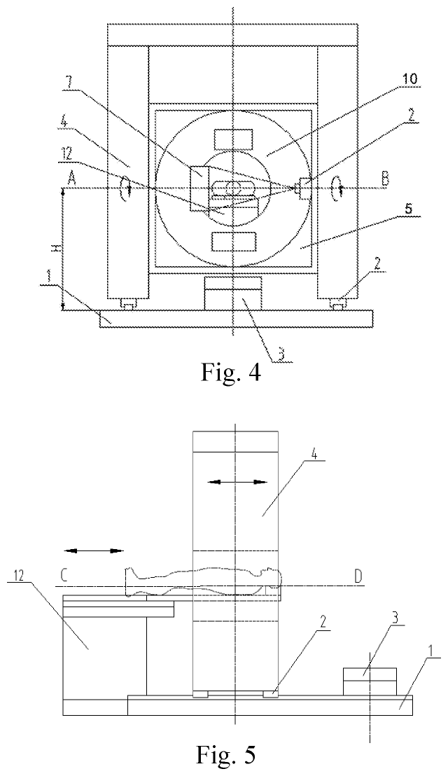

As shown in FIGS. 3, 4 and 5, after entering the basic patient information, the operator selects the 3D scanning of the patient in the lying position, and the system will switch to the lying-position scanning mode. The scanning frame 5 is turned by 90 degrees at a height H to the state shown in FIG. 4, and the scanning bed 12 is lowered to the lowest initial position.

The patient is correctly lying on the scanning bed 3 with the confirmation of the auxiliary system, and the auxiliary system is used to define the required scanning examination range. The examination is started, and the apparatus runs according to the logical actions required by the control system. The scanning bed 12 is raised to a predetermined height and is moved forward to the scanning start position. The rotary scanning table 10 rotates, and the bulb tube 6 emits a beam as required. The dynamic flat-panel detector 7 receives the projection information. The control system transmits data for saving. After scanning for one cycle, the scanning bed moves forward by one imaging height for the next scanning, until the defined segments are scanned, and the machine returns to the initial state. Then the patient leaves the scanning bed. After the data processing system processes the data to generate a data file, the operator outputs the corresponding data, which can be electronic data or film, according to the examination requirements.

### Embodiment 3

2D scanning and imaging of a patient:

During the scanning process of the embodiment 1 and the embodiment 2, the rotary scanning table 10 performs static imaging only in the normal position and the lateral position to complete scanning and imaging of an axial imaging range of human body, and the DR imaging both in the standing state and in the lying state can be realized.

The cone-beam CT multi-directional scanning apparatus provided by the invention makes it possible to obtain a three-dimensional bone imaging data on a human body's upright position which cannot be realized by the current scanning apparatus. Three-dimensional cone-beam CT imaging and two-dimensional DR forming can be implemented on both standing position and lying position, so that three-dimensional images of human bone system and soft tissues with high spatial precision and high density precision can be acquired, thereby providing good support for clinical biomechanical evaluation, diagnosis, treatment, surgical plan formulation, and simulation.

The above only gives some embodiments of the present invention which is not intended to be limited by these embodiments. Other variations and modifications made by those skilled in the art without departing from the inventive concept shall fall within the scope of the present invention. Therefore, the scope of protection of the invention shall be defined by the attached claims.

1. A cone-beam CT multi-directional scanning apparatus for whole body scanning and imaging of a patient, comprising a host machine frame, a scanning frame, a revolving driving device, a lifting platform provided on two sides of the host machine frame and a cone-beam CT scanning device arranged on a body of the scanning frame, wherein a lifting end of the lifting platform is connected to the scanning frame by virtue of the revolving driving device so that the scanning frame can turn over inside the host machine frame, and wherein with the turning of the scanning frame, when a working surface of the cone-beam CT scanning device on the body of the scanning frame is in a horizontal state, scanning and imaging of a patient in a standing position can be performed, and when the working surface of the cone-beam CT scanning device on the body of the scanning frame is in a vertical state, scanning and imaging of the patient in a lying position can be performed.

2. The cone-beam CT multi-directional scanning apparatus according to claim 1, wherein the rotary axis of the scanning frame and of the lifting platform and the beam center axis are at the same height.

3. The cone-beam CT multi-directional scanning apparatus according to claim 1, wherein the lifting platform can drive the scanning frame to move up and down.

4. The cone-beam CT multi-directional scanning apparatus according to claim 1, wherein the cone-beam CT scanning device comprises a rotary scanning table, a bulb tube, and a dynamic flat-panel detector, wherein a detection port is provided in the middle of the rotary scanning table, the rotary scanning table is arranged on the body of the scanning frame, and the bulb tube and the dynamic flat-panel detector are oppositely disposed on both sides of the detection port.

5. The cone-beam CT multi-directional scanning apparatus according to claim 4, wherein the cone-beam CT scanning device is disposed on the rotary scanning table.

6. The cone-beam CT multi-directional scanning apparatus according to claim 1, wherein the revolving driving device comprises a revolving axis, a bearing, and a motor, wherein a power end of the revolving axis is connected to an output end of the motor, and the bearing is sleeved outside the revolving axis.

7. The cone-beam CT multi-directional scanning apparatus according to claim 1, further comprising a base and a main base guide rail pair, wherein the base is connected to the host machine frame through the main base guide rail pair, so that the host machine frame slides along the main base guide rail pair.

8. The cone-beam CT multi-directional scanning apparatus according to claim 7, wherein the sliding of the host machine frame along the main base guide rail pair comprises a front and rear movement of the host machine frame along a guide rail of the main base guide rail pair under the drive of the motor.

9. The cone-beam CT multi-directional scanning apparatus according to claim 4, further comprising a standing platform, wherein the standing platform is disposed below the detection port.

10. The cone-beam CT multi-directional scanning apparatus according to claim 9, wherein the standing platform is a liftable platform so as to cooperate with the lifting platform connected to the scanning frame.

11. The cone-beam CT multi-directional scanning apparatus according to claim 4, further comprising a lifting scanning bed, wherein the width of the lifting scanning bed is smaller than the diameter of the detection port.

12. The cone-beam CT multi-directional scanning apparatus according to claim 11, wherein the lifting scanning bed is independently placed at the rear of the host machine frame.

13. The cone-beam CT multi-directional scanning apparatus according to claim 11, wherein a horizontal slide rail which extends through the rotary scanning table in the vertical state is provided below the lifting scanning bed, so that the lifting scanning bed can slide in a horizontal direction.
